(19) United States
(12) Patent Application Publication
(10) Pub. No.: US 2007/0134280 A1
(43) Pub. Date: Jun. 14, 2007

(54) Thixotropic ingestible formulation to treat sore throat

(76) Inventors: Stephen B. Roman, Phoenix, AZ (US); Gary S. Kehoe, Phoenix, AZ (US)

Correspondence Address: TOD R NISSLE, PO BOX 55630, PHOENIX, AZ, 85078, US

(21) Appl. No.: 11/704,628

(22) Filed: Feb. 9, 2007

Related U.S. Application Data
(63) Continuation-in-part of application No. 11/501,609, filed on Aug. 9, 2006.
Application No. 11/501,609 is a continuation-in-part of application No. 11/210,293, filed on Aug. 24, 2005.
(60) Provisional application No. 60/635,095, filed on Dec. 10, 2004.
(60) Provisional application No. 60/772,398, filed on Feb. 10, 2006.

(51) Int. Cl. A61K 9/00; A61K 31/715

(52) U.S. Cl. 424/400; 514/54

(57) ABSTRACT

A thixotropic formulation includes a high molecular weight, thixotropic, gel-forming, naturally occurring polysaccharide extracted from algae and comprised of repeating sulfated and non-sulfated galactose and 3,6 anhydrogalactose (3,6-AG) units, and includes water.

This application is a continuation-in-part of application Ser. No. 11/501,609, filed Aug. 9, 2006, which is a continuation-in-part of application Ser. No. 11/210,293 filed Aug. 24, 2005 and also derives from and claims priority based upon provisional patent application Ser. No. 60/635,095 filed Dec. 10, 2004 and provisional patent application Ser. No. 60/772,398, filed Feb. 10, 2006.

This invention pertains to formulations for epithelial tissue layers, especially mucosal tissue, which formulations can provide lubricity, provide protection from disease, deliver medicaments, and exhibit a viscosal stability sufficient to maintain the formulations in contact with mucosal tissue for extended periods of time. The invention also pertains to methods of preparing, dispensing and using such formulations in accordance with the invention.

More particularly, the invention pertains to ingestible formulations to treat, to moisturize, and to deliver medicants to epithelial tissues in the throat and to deliver medicants to the gastro intestinal tract.

Conventional formulations, once applied to epithelial tissues, often tend to have little viscosity, tend to lose their viscosity and travel over and away from desired epithelial tissues at a faster than desired rate, or, in the manner of ZICAM (TM) gel tend to dry up and leave behind a cellulose mass, and to not coat and maintain contact with desired areas in the throat. Consequently, it is difficult to apply such formulations and to have the formulations remain in position for an extended period of time. In addition, such formulations can contain artificial compositions, can permit movement of macrophages through the formulation, and can be inconvenient to apply.

The use of a chilled or frozen formulation to deliver a medicant is desirable because as the temperature of a composition is reduced, the disagreeable taste associated with a particular medicant tends to diminish or be blunted. When, however, a medicant is intended to be applied to the throat of a patient, a frozen popsicle-type composition is not desirable because in a user's mouth it become water and quickly bypasses the throat on the way to the gastrointestinal tract. In addition, popsicle-type composition are not well-suited to surviving a thaw-freeze cycle. Such a freeze thaw cycle can occur during transport from a grocery store to home, or can occur when the item is only partially consumed and returned to a freezer. Further, popsicle-type compositions can not readily be stored in an above-freezing stored state and can not be stored a room temperature during transport from a manufacturer to a good store.

It would be desirable to provide a formulation that would maintain contact with epithelial tissues for an extended period of time, that could be ingested and coat desired areas of the throat, that would not dry up and would remain moist, that inhibit the passage of macrophages and other harmful bodies through the formulation, that would not contain artificial compositions, that would be pleasant tasting, that could be chilled or frozen, that would reduce the risk of choking when ingested, and that would permit shear induced movement of formulation to facilitate travel of the formulation down the throat and onto sore and infected areas in the throat.

It would also be desired to provide a formulation and method that would provide an ingestible composition that would survive freeze-thaw-freeze or chill-warm-chill cycles with its physical properties intact, that would ameliorate the bitter taste of some medicants, that would have a consistency that was soothing to the mouth of a user, that would facilitate the application of medicants to a sore throat, and that would facilitate the ingestion of medicants by children and other individuals that have an aversion to ingesting pills, cough syrup, and other common medicinal products.

We have discovered an improved ingestible formulation for relieving discomfort in the throat and for delivering medicants to the gastro intestinal track of an individual. The formulation comprises a thixotropic gel having a viscosity at room temperature in the range of 2,000 to 80,000 centipoise, preferably 5,000 to 30,000 centipose; having a pH in the range of 2.6 to 10; and, comprising naturally occurring compositions. The formulation preferably includes natural components including a high molecularweight, thixotropic, gel-forming, naturally-occurring polysaccharide extracted from algae and composed of repeating sulfated and non-sulfated galactose and 3,6 anhydrogalactose (3,6-AG) units; and, water. The formulation inhibits the passage of macrophages through the composition, is natural, and retains moisture. Desired medicants or other components can be incorporated in the formulation. Formulations prepared in accordance with the invention can be utilized when the formulations are room temperature, when the formulations are chilled, or when the formulations are frozen.

One embodiment of the invention comprises a method of relieving discomfort in a patient's throat. The method comprises the step of providing a frozen personal lubricant thixotropic formulation to dispense multiple equivalent doses of the formulation. The frozen formulation has a viscosity in the range of 5,000 to 80,000 centipoise, has a pH in the range of 2.6 to 10, and comprises water and a high molecularweight, thixotropic, gel-forming, naturally-occurring polysaccharide extracted from algae and composed of repeating sulfated and non-sulfated galactose and 3,6 anhydrogalactose (3,6-AG) units. The method also includes the steps of ingesting the frozen formulation in the patient's mouth; and, allowing shear produced by movement of the mouth to reduce the viscosity of the thixotropic formulation and facilitate movement of some of the formulation from the mouth to the individual's throat. Depending on the desired degree of thixotropicity in the frozen formulation or in another formulation in accordance with the invention, the amount of a polysaccharide can be adjusted. In the event two or more polysaccharides are utilized in the frozen formulation or in another formulation in accordance with the invention, the ratio of one polysaccharide with respect to another polysaccharide can be adjusted.

The particular polysaccharide presently utilized in the formulations of the invention is critical. Although a multitude of polysaccharides exist, the critical polysaccharide utilized in the invention is a high molecular weight thixotropic polysaccharide made up of repeating galactose and 3,6 anhydrogalactose (3,6-AG) units, both sulfated and nonsulfated and extracted from algae, typically Eucheuma, Chondrus, Chondus crispus, and Gigartina red benthic marine algae that are multicellular and macrothallic. Compositions comprising this polysaccharide and water can, advantageously, be chilled or frozen. As used herein, a polysaccharide is thixotropic when it produces a thixotropic solution or gel when admixed with water or another liquid.

Three specific types of high molecular weight galactose polysaccharides extracted from marine algae are kappa, iota, and lambda.

Kappa polysaccharide typically forms a strong, rigid aqueous gel; has some syneresis; and, forms a helix with potassium ions. Calcium ions cause the helices in kappa formed gel to aggregate and cause the gel to contract and become brittle. Gel formed with kappa polysaccharide is slightly opaque, but becomes clear when sugar is added. Kappa polysaccharide is about 25% ester sulfate and about 34% 3,6-AG.

Iota polysaccharide forms an elastic aqueous thixotropic gel and forms a helix with calcium ions. Limited aggregation in iota formed gel contributes to the elasticity of the gel. There is no syneresis. The gel is clear. When iota formed gel is frozen and thawed, its viscosity remains stable, as generally do gels formed with iota polysaccharide in combination with kappa polysaccharide and/or lambda polysaccharide. Iota polysaccharide is about 32% ester sulfate and 30% 3,6-AG.

Lambda polysaccharide does not form an aqueous gel. Lambda polysaccharide is about 35% ester sulfate and includes little or no 3,6-AG.

While lambda and kappa polysaccharide can be utilized alone, in combination with each other, or in combination with iota polysaccharide in producing formulations utilized in the invention, when a thixotropic formulation is desired-which is the case in the presently preferred embodiment of the invention-iota polysaccharide must be utilized. Iota polysaccharide presently comprises at least 25%, preferably at least 33%, most preferably at least 50% of a quantity of high molecular weight galactose algae polysaccharide utilized to prepare a batch or quantity of the formulation in accordance with the invention. The remaining portion of the quantity of galactose algae polysaccharide used to prepare a batch of the formulation can comprise lambda or kappa polysaccharide. When solids are admixed with water to produce the formulation, the high molecular weight galactose algae polysaccharide comprises at least 50%, preferably at least 75%, most preferably at least 80% of the solids, while water or other liquids comprise the remainder of the composition.

The concentration of high molecular weight galactose polysaccharide in formulations of the invention is in the range of 0.1% to 8.0% by weight, preferably in the range of 1% to 4% by weight, most preferably in the range of 1.5% to 3.5% by weight. As noted, the galactose polysaccharide can consist of iota, lambda, and/or kappa polysaccharide.

If the formulation of the invention includes 0.5% by weight of iota galactose polysaccharide, thixotropic properties are not apparent. If the formulation includes 0.75% by weight iota polysaccharide, some thixotropic properties are evidenced. 1.0% by weight of iota polysaccharide provides more evidence of thixotropic properties; 1.5% by weight provides good evidence; and, when there is 1.75% by weight iota polysaccharide the thixotropic property of the gel formulation is very noticeable. Consequently, it is preferred that the formulation of the invention include at least 1.0% by weight iota polysaccharide, preferably at least 1.5% by weight iota polysaccharide, and most preferably at least 1.75% by weight iota polysaccharide. Lesser fractions of lambda and kappa polysaccharides are normally, but not necessarily, included with iota polysaccharide.

Iota, kappa, and lambda polysaccharides are sold by various sources, including FMC Corporation, 1735 Market Street, Philadelphia, Pa. 19103, and CP Kelco, 311 S, Wacker Drive, Suite 3700, Chicago, Ill. 60606. Examples of galactose polysaccharides sold by FMC Corporation are 373/Gelcarin GP 911 [Kappa polysaccharides comprise at least majority of composition], 335/Gelcarin GP 379 [Iota polysaccharides comprise at least majority of composition], 303/Gelcarin GP 812 [Kappa polysaccharides], 205/Viscarin GP 109 [Lambda polysaccharides], 201Niscarin GP 209 [Lambda polysaccharides], and, 357/Seaspen PF [Iota polysaccharides, phosphates, CaSO4-2H20]. Examples of galactose polysaccharides sold by CP Kelco are Genuvisco type X-931-03 (CP Kelco), and Genuvisco type X-923-03 (CP Kelco) [Iota polysaccharides].

Minor effective amounts of preservatives, typically in the range of 0.01% to 1.0% by weight, can be included in the formulation. By way of example, and not limitation, methylparaben, propylparaben, potassium sorbate, and benzoic acid are common preservatives than can be utilized.

Effective amounts of appropriate acidic or basic compositions can be included in the formulation to adjust and control pH in the desired range of 2.6 to 10, preferably in the range of 4.0 to 7.0. The presently preferred pH is 5.5. By way of example, and not limitation, citric acid and sodium hydroxide comprise compositions commonly utilized to adjust the pH of the personal lubricant.

Minor effective amounts of flavoring, topical stimulants (i.e., to produce a warming or cooling sensation) coloring, or odor producing compositions (i.e., fragrances) can be incorporated in the formulation in either a liquid, solid or gaseous form or mixture thereof.

The water utilized preparing the personal lubricant can be de-ionized water, USP water, de-chlorinated water, mineral water, water treated with activated carbon, tap water, etc. Naturally occurring oils or other fluids can, if desired, be utilized in place of or in combination with water.

Dextromethorphan, anesthetics, or any other desired medicant can be incorporated in the formulation. Formulations or medicants can be encapsulated or provided in other time release forms.

Dosage can vary per the user's discretion, but the volume of a single dose typically is in the range of 0.1 mL (milliliter) to 15 mL, preferably 1.0 mL to 6.0 mL.

The following examples are given by way of illustration and not limitation of the invention.

### EXAMPLE I

The following ingredients are provided.

| Ingredient | Weight Percent |
|---|---|
| Polysaccharide | 4.5 |
| Iota polysaccharide | 3.0 |
| Kappa polysaccharide | 0.75 |
| Lambda polysaccharide | 0.75 |
| Glycerin USP Natural | 3.00 |
| Sodium Hydroxide (pH adjustment) | .05 |
| Citric Acid (pH adjustment) | .05 |
| Methylparaben (preservative) | .10 |
| Propylparaben (preservative) | .10 |
| Dextromethorphan HBr (active) | .03 |
| De-ionized water | 92.17 |

The water, polysaccharides, sodium hydroxide, and glycerin are admixed under agitation to the water at room temperature. The pH of the resulting aqueous formulation is adjusted to 5.5 by adding the citric acid. The preservatives and dextromethorphan HBr are added while the aqueous formulation is stirred. Care is taken to avoid entrainment of air when the aqueous formulation is stirred or otherwise agitated. The resulting formulation has a viscosity of 5,500 centipoise. The viscosity of formulations in accordance with the invention is, as noted, in the range of 3,000 cps to 80,000 cps, but a viscosity in the range of 5,000 to 30,000 cps is preferred. A quantity of the formulation is charged in a container. The container selected is configured to dispense 1.0 mL of the formulation each time the container is utilized. The container can be operated to deliver the exact amount of formulation (dosage) prescribed. Once such container is produced by Mega Pumps L.P. of 611 Industrial Way West, Eatontwon, N.J. 07724. Gel capsules or any other desired container can be utilized to dispense a metered amount of the formulation. A particular desired feature of a container produced by Mega Pumps is that the container can dispense formulation when the container is in any orientation. Further, the container prevents the admixing of air with the formulation when the gel is dispensed. The container is preferably designed such that the container can—preferably with only a single hand—be grasped and manipulated by a user to dispense a dose of formulation from a nozzle on the container directly into a user's mouth. The nozzle can be configured to be inserted a selected distance in a user's mouth prior to operating the container to select a metered dose of formulation. Movement of the mouth, and therefore the formulation in the mouth, generates shear acting on the formulation and causes the formulation to liquify and spread out over the contact surface areas in both the mouth and throat.

### EXAMPLE II

Example I is repeated except that instead of 3% by weight of iota polysaccharide and 0.75% of lambda polysaccharide being utilized, 1.5% by weight of iota polysaccharide is utilized and 1% by weight of lambda polysaccharide is utilized. Similar results are obtained.

### EXAMPLE III

Example I is repeated except that instead of 3% by weight of iota polysaccharide, 0.75% by weight of iota polysaccharide, and 0.75% by weight of kappa polysaccharide being utilized, 3.0% by weight of iota polysaccharide is utilized. Similar results are obtained.

### EXAMPLE IV

Example I is repeated except that, 0.65% of sodium hydroxide is utilized to adjust the pH to 8.0 instead of 5.5. Similar results are obtained.

### EXAMPLE V

3.0 mL of the formulation of EXAMPLE I is dispensed into the mouth of a woman. Shear produced by movement of the individual's mouth causes the formulation to flow and coat epithelial tissues in the mouth and throat of the individual.

### EXAMPLE VI

The composition of Example I is frozen. 3.05 mL of the frozen formulation is dispensed in the mouth of a man. Shear and heat produced by movement of the individual's mouth cause the formulation to flow and coat epithelial tissues in the mouth and throat of the individual.

### EXAMPLE VII

The following ingredients are provided.

| Ingredient | Weight Percent |
|---|---|
| Iota Polysaccharide (Genuvisco X-931-03) | 3.0 |
| Polyol (Xylitol) | 3.0 |
| Pectin (Gum Pecting) | 1.5 |
| Flavoring (Art. Cherry Flavor | 0.1 |
| # 825.286/WC) | |
| Flavoring (Nat. & Art. Vanilla Flavors | 0.85 |
| #F86878) | |
| Flavoring (Chocolate Flavor N&A | 0.85 |
| #826.720/WC) | |
| Drug (Dextromethorphan Hbr. USP) | 0.035 |
| High Intensity Sweetener (Acesulfame-K, BP) | 0.050 |
| Colorant (CSL33665 Red Lake | 0.125 |
| Disperson “R” G.B.) | |
| High Intensity Sweetener (Suryl | 0.6 |
| Sucralose Liquid) | |
| Humectant (Glycerin, USP Natural) | 3.0 |
| Sensate-cooling (Menthol Crystals | 0.3 |
| Preservative (Potassium Sorbate) | 0.1 |
| De ionized water | 86.76 |

The water and dextromethorphan are admixed at room temperature to produce an aqueous mixture. The xylitol and red lake dispersion are admixed to the aqueous mixture, after which the glycerin is admixed to the aqueous mixture to produce a uniform aqueous mixture. The potassium sorbate is slowly admixed to the aqueous mixture, after which the acesulfame-K and sucralose liquid are admixed to the aqueous mixture. The cherry flavor, vanilla flavor, chocolate flavor and menthol crystals are premixed together to form a premix composition. One half of the Genuvisco and of the pectin solution are added to the aqueous solution along with the premix composition. The pH is measured and is 4.75. The remainder of the Genuvisco and pectin solution are admixed to the aqueous solution to produce the completed formulation. At room temperature, the completed formulation has a pudding-like consistency. One advantage of the completed formulation and other formulations utilizing iota polysaccharides with or without lambda and kappa polysaccharides is that the formulations can experience two or more freeze-thaw-freeze cycles without any apparent change or only a minimal change in the physical properties of the formulations. This is believed to be the case because the formulations “hold on to” water and do not permit water to separate out and freeze.

In one embodiment of the invention, the completed formulation of Example VII (or other thixotropic polysaccharide formulations prepared in accordance with the invention) is frozen, in, by way of example and not limitation, an elongate pliable plastic container that, when one end of the container is opened, permits the frozen composition to be squeezed out through the top of the container. One advantage of this kind of packaging is that it facilitates eating the formulation of the invention, even if the formulation is not frozen and is only chilled or is at room temperature. Another advantage of formulations prepared in accordance with the invention is that they can be stored and dispensed at room temperature or can be stored and dispensed chilled or frozen. Further, packaging the formulation in popsickle-like plastic containers of the type described often facilitates the ingestion of a medicant because (1) chilling or freezing a medicant typically abates a bitter taste the medicant may have, (2) the volume of the container (i.e., the volume of the formulation in the container) typically is significantly greater than the volume of a tablespoon of cough syrup, of a pill, or of other conventional medicine administering substances or configurations, which means that one dose of a medicant in a container of the formulation of the invention is distributed throughout a significantly greater volume and that the resulting weaker concentration of the medicant in the formulation of the invention minimizes a bitter taste the medicant may have, and (3) as the frozen formulation is heated and sheared in the mouth of a patient, it preferably has or takes on the consistency of pudding, which consistency is, for most patients, pleasing. The container preferably has a “child-proof” lid or closure mechanism, or, the container is placed and stored (prior to use) in a second container that has a “child-proof” closure mechanism. The use of a “child-proof” closure mechanism is important because a formulation produced in accordance with the invention typically, although not necessarily, will have a pleasing taste and children may want to consume more than one container of the formulation. Since the formulation may include doses of medicants, vitamins, minerals, or other desired substances that should only be ingested in limited quantities, it is important to reduce the risk that an unsupervised child can readily ingest multiple containers of the formulation.

When frozen, the completed formulation of Example VII has a non-rigid, clay-like consistency and can be plastically deformed. The formulation when frozen has little, if any, resiliency. The concentration or amount of polysaccharides in the formulation can be adjusted as desired to attain such a consistency when frozen. Shear and heat reduce the viscosity of the formulation.

The iota, kappa, and lambda polysaccharides utilized in some of the formulations of the invention are sensitive to cations like sodium, potassium, and magnesium. The sodium cation increases the viscosity of the polysaccharides. It is therefore, in one embodiment of the invention, preferred to utilized a composition or component that functions as a fragrance and preservative that includes a sodium, potassium, and/or magnesium ion. The concentration of the fragrance—preservative composition that is in the finished formulation is in the range of 0.01% to 3.0% by weight. On such preferred fragrance—preservative composition comprises sodium phytate. Another such composition comprises sodium benzoate.

When glycerin is utilized in the formulation, it is preferably 97.5% glycerin, with the remainder of the glycerin being water. Some “glycerin” compositions are 60% to 70% by weight glycerin, with the remainder being water. Using glycerin compositions that are only 60% to 70% glycerin, although possible, is not presently preferred because the polysaccharides will tend to clump into balls when they contact the water in the glycerin. Glycerin compositions that are at least 90% by weight glycerin are preferred in the practice of the invention because the polysaccharides tend during mixing to disperse uniformly in such glycerin compositions and tend not to clump.

It is possible to prepare formulations of the invention in the form of a syrup, a lozenge, a thin film, or a spray.

A syrup is similar to a gel but has a lower viscosity in the range of 600 cps to 4,000 cps.

A lozenge is a formulation of the invention made with a water content of less than two percent by weight. The lozenge is made with high shear equipment, extrusion, or other similar equipment to produce a lozenge that dissolves slowly and produces a liquid film that will in turn coat the mouth and throat, preventing transdermal migration of macrophages and also optimally delivering other medicant to the user.

A thin film is similar to a lozenge, but has moisture content of less than 0.75% by weight.

A spray is similar to a syrup and has a viscosity in the range of 400 cps to 4,500 cps.

When a thixotropic formulation is not required, and when the loss of water during freeze-thaw cycles is not a concern, zanthan, guar, carbomethylcellulose, methylethylcellulose, and other gums or thickeners can be utilized in combination with or in place of the presently preferred naturally-occurring polysaccharide that is extracted from algae and comprises repeating sulfated and non-sulfated galactose and 3,6 anhydrogalactose (3,6-AG) units. It is also believed possible to produce thixotropic formulations by admixing selected gums or thickeners.

Having described our invention in such terms as to enable those of skill in the art to make and practice it, and having described the presently preferred embodiments thereof, We Claim:

1. A method of orally delivering at least one medicant, comprising the steps of
(a) providing a thixotropic, viscous formulation that
(i) at room temperature has a viscosity in the range of 2,000 cps to 80,000 cps,
(ii) when frozen at thirty degrees F high molecular weight is non-rigid, plastic, and deformable,
(iii) when subjected to a freeze-thaw-freeze cycle in which the temperature of said formulation moves from thirty degrees to seventy-six degrees to thirty degrees, substantially retains viscosity and plastic deformability physical properties that said formulation had prior to the freeze-thaw-freeze cycle, and
(iv) includes a minor effective amount of at least one selected medicant;
(b) placing a selected quantity of said formulation in the mouth of a patient; and,
(c) allowing shear produced by movement of the patient's mouth to reduce the viscosity of the formulation to facilitate movement of at least some of the formulation over epithelial tissue in the individual's throat and down the esophagus of the patient.

2. The method of claim 1 wherein said formulation includes water and a minor effective amount of a, thoxitropic, gel-forming, naturally-occurring polysaccharide extracted from algae and composed of repeating sulfated and un-sulfated galactose and 3,6 anydrogalactose (3,6-AG) units.

3. The method of claim 1 wherein said formulation has a viscosity in the range of 3,000 to 80,000 centipoise.

4. The method of claim 2 wherein said formulation has a viscosity in the range of 3,000 to 30,000 centipoise.

5. The method of claim 1 wherein said formulation is frozen prior to being placed in the patient's mouth.

6. The method of claim 2 wherein said formulation is frozen prior to being placed in the patient's mouth.

7. The method of claim 1 wherein the quantity of medicant per unit volume of said formulation is less that the quantity of medicant per unit volume in at least one other commonly administered composition that contains said medicant.

8. The method of claim 1 wherein the quantity of medicant per unit volume of said formulation is less than the quantity of medicant per unit volume of cough syrup.

9. The method of claim 2 wherein the quantity of medicant per unit volume of said formulation is less than the quantity of medicant per unit volume of cough syrup.

10. The method of claim 2
(a) wherein said polysaccharide is iota polysaccharide; and,
(b) including the additional step of freezing said formulation prior to said formulation being placed in the mouth of the patient.

11. The method of claim 10 wherein the quantity of medicant per unit volume of said formulation is less than the quantity of medicant per unit volume of cough syrup.

12. The method of claim 10 including the additional steps of
(a) prior to freezing said formulation, placing said formulation in a container with a child proof lid; and,
(b) after freezing and prior to placing said formulation in the mouth of a patient, opening said child proof lid.

13. The method of claim 2
(a) wherein said polysaccharide is iota polysaccharide; and,
(b) including the additional steps of
(i) prior to placing said formulation in the mouth of a patient, packaging said formulation in a container with a child proof lid, and
(ii) opening said child proof lid.
